(19) United States
(12) Patent Application Publication
(10) Pub. No.: US 2007/0049952 A1
(43) Pub. Date: Mar. 1, 2007

(54) APPARATUS AND METHOD FOR MITRAL VALVE REPAIR WITHOUT CARDIOPULMONARY BYPASS, INCLUDING TRANSMURAL TECHNIQUES

(76) Inventor: Steven J. Weiss, Haverford, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/463,675

(22) Filed: Aug. 10, 2006

Related U.S. Application Data
(60) Provisional application No. 60/712,879, filed on Aug. 30, 2005.

(51) Int. Cl. A61B 17/04

(52) U.S. Cl. 606/144

(57) ABSTRACT

A method and apparatus for repairing the heart's mitral valve by using anatomic restoration without the need to stop the heart, use a heart-lung machine or making incisions on the heart. The method involves inserting a leaflet clamp through the heart's papillary muscle from which the leaflet has been disconnected, clamping the leaflet's free end and then puncturing the leaflet. One end of a suture is then passed through the hollow portion of the clamp, while the other end of the suture is maintained external to the heart. The clamp is then removed and the suture's two ends are fastened together with a securement ring/locking cap assembly to the heart wall exterior, thereby reconnecting the leaflet to the corresponding papillary muscle. The introduction of the clamp, puncturing of the leaflet, passage of the suture therethrough and removal of the clamp can be conducted a plurality of times before each suture's two ends are fastened to the securement ring/locking cap assembly.

### CROSS-REFERENCE TO RELATED APPLICATIONS

This utility application claims the benefit under 35 U.S.C. §119(e) of Provisional Application Ser. No. 60/712,879 filed on Aug. 30, 2005 entitled APPROACH TO AND DEVICE FOR MITRAL VALVE REPAIR WITHOUT CARDIOPULMONARY BYPASS AND EVENTUALLY WITHOUT AN INCISION WITH THORACOSCOPIC TECHNIQUES and whose entire disclosure is incorporated by reference herein.

### BACKGROUND OF THE INVENTION

1. FIELD OF INVENTION

This invention relates to the mitral valve of the heart and more particularly, to methods and apparatus for repairing flail mitral valve leaflets.

2. DESCRIPTION OF RELATED ART

As shown most clearly in FIG. 1, the mitral valve 2 of the heart 3 comprises leaflets 4A and 4B that are attached to corresponding papillary muscles 5A and 5B through respective chordae tendinae 6A and 6B; thus, the chordae tendineae tether the mitral leaflet. FIG. 1 depicts a damaged mitral valve 2 in that one of leaflets 4B has flailed, e.g., the chordae tendineae 6B have ruptured, thereby separating the leaflet 4B from the papillary muscle 5B. This causes the now unsupported leaflet 4B to flail and the mitral valve 2 to leak and is referred to as “flail mitral valve” or just “flail.”

The majority of routine mitral valve repairs presented in common clinical practice in the United States involves a flail mitral valve leaflet, typically, the P2 scallop of the posterior leaflet but it should be understood that other leaflet segments may be involved as well. Conventional surgical repair techniques have evolved from the work of Dr. Alan Carpentier, and typically involve resecting the unsupported or flail portion of the leaflet, which then requires reducing the size of the mitral annulus with application or suture shortening, leaflet repair with either primary or sliding plasty and implantation of a re-enforcing annulus ring. Previous approaches all involved connecting the patient to the heart lung machine to be able to safely stop the heart and approach the mitral valve by making an atrial incision. Recently, equivalent success rate and long term durability has been achieved with implantation of artificial chordae typically using 4-0 or 5-0 Gortex suture that has achieved equivalent success and long term durability measures. Some authors have reported finite element stress measurements on the repaired leaflet and note that conventional techniques flatten the leaflet or reduce its saddle shape creating more leaflet stress. They predict better durability with chordal replacement than conventional leaflet resection. However, both of these techniques require open visualization of the mitral valve with an arrested heart.

In addition, the devices being used in this type of mitral valve repair must minimize the use of small components, including fasteners, that can accidentally dislodge from the device or instrument or completed repair, and cause an embolism.

Thus, there remains a need for a new method and apparatus for supporting the leading edge of the flail leaflet segment with artificial chordae to the corresponding papillary muscle tip (e.g., posterior papillary muscle tip) that can be accomplished by a trained cardiothoracic surgeon monitoring the beating heart without cardiopulmonary bypass and, ideally, without an incision, e.g., using thoracoscopic techniques.

All references cited herein are incorporated herein by reference in their entireties.

### BRIEF SUMMARY OF THE INVENTION

A method for repairing the mitral valve of a heart wherein at least one leaflet has suffered a defect with respect to its papillary muscle (e.g., a flail leaflet has partially detached, suffered chordal rupture or chordal defect such as but not limited to, elongated chordal defect) . The method comprises: introducing a clamp transmurally into the beating heart and through the papillary muscle; grasping a portion of the leaflet with the clamp; piercing a hole in the leaflet; inserting a suture, having a first end, through the clamp and through the hole, and wherein the first end is displaced through the clamp instrument to emerge from a proximal end of the clamp; removing the clamp from the beating heart; and securing the first end, and a second end, of the suture against an exterior wall of the beating heart.

A method for repairing the mitral valve of a heart wherein at least one leaflet has suffered a defect with respect to its papillary muscle (e.g., a flail leaflet has partially detached, suffered chordal rupture or chordal defect such as but not limited to, elongated chordal defect). The method comprises: (a) introducing a clamp transmurally into the beating heart and through the papillary muscle; (b) grasping a portion of the leaflet with said clamp; (c) piercing a hole in the leaflet; (d) inserting a suture, having a first end, through said clamp and through said hole, said first end being displaced through said clamp to emerge from a proximal end of said clamp; (e) maintaining a second end of said suture external to the beating heart; (f) removing said clamp from the beating heart; (g) repeating steps (a)-(f) to establish a plurality of first ends that emerge from a proximal end of said clamp and a plurality of second ends that are maintained external to beating heart; and (h) securing the plurality of first ends and the plurality of second ends against an exterior wall of the beating heart.

An apparatus for repairing the mitral valve of a heart wherein at least one leaflet has suffered a defect with respect to its papillary muscle (e.g., a flail leaflet has partially detached, suffered chordal rupture or chordal defect such as but not limited to, elongated chordal defect). The apparatus comprises: a clamp comprising first and second elongated members having respective first and second distal ends for clamping the leaflet; an external cylinder in which the clamp is slidable; a hollow piercing member, having a leading edge that can pierce tissue, that slides within the clamp; a suture driver device that couples to one end of the hollow piercing member, and wherein movement of the external cylinder acts on the first and second members to open or close the clamp to grasp or release the leaflet, and wherein the displacement of the hollow piercing member punctures the leaflet to form a hole therein and wherein the suture driver device drives a suture through the hollow piercing member for permitting said suture to pass through the leaflet and through the clamp for supporting mitral valve repair by connecting the leaflet to the papillary muscle.

An apparatus for stabilizing a portion of the heart wall of a beating heart to permit the transmural introduction of surgical instruments through the heart. The apparatus comprises a housing having: a first support surface that contacts the heart wall of the beating heart and provides a stable target for transmural penetration; a central passageway for permitting coupling of an epivascular ultrasound probe, for the passage of instruments used for the Seldinger technique, and for the passage of an introducer therethrough; and an extension formed with the first support surface for coupling to an externally fixed object.

A suture driver device for driving a suture through a surgical device that has penetrated some portion of a living being and wherein the surgical device provides a path for delivery of the suture. The suture driver device comprises: a syringe having a port that can couple to the surgical device, wherein the syringe comprises a chamber filled with a biocompatible fluid and with the suture, and wherein the suture comprises a weighted end that is initially disposed at the port such that when said syringe is activated, the weighted end is driven through the surgical device.

A suture driver device for driving a suture through a surgical device that has penetrated some portion of a living being and wherein the surgical device provides a path for delivery of the suture. The suture driver device comprises a syringe having: a first port that can couple to a proximal end of the surgical device; a second port, in fluid communication with the first port; a chamber in fluid communication with the first and second ports and filled with a biocompatible fluid; and wherein, before the suture driver device is activated, the suture is passed through the first port and through the second port so that a first end of the suture is located externally of the suture driver device and a first weighted end of the suture is positioned at the first port.

An apparatus for securing the free ends of a suture that have passed through an internal body part of a living being. The apparatus comprises: a ring having an inner surface with a plurality of channels, wherein each of the channels comprises teeth; a corresponding plug that fits snuggly within an opening of the ring; wherein the free ends of the suture are passed through the opening in the ring and each one of the free ends are positioned in respective ones of the plurality of channels and wherein the plug is then positioned snuggly within the opening and wherein the ring and plug are positioned against the internal body part.

A strain gauge device for detecting the tension applied to the free ends of a suture that has passed through the body part of a living being. The strain gauge device comprises a housing that can be coupled to the free ends of the suture and wherein the housing comprises: a strain gauge or load cell for detecting the strain or load applied to the suture; a display, coupled to the strain gauge or load cell, for displaying tension values; a stepper motor, coupled to the display, for increasing or decreasing applied tension to the suture; and control keys coupled to the display and to the stepper motor for permitting a user to control the tension applied to the suture.

### BRIEF DESCRIPTION OF SEVERAL VIEWS OF THE DRAWINGS

The invention will be described in conjunction with the following drawings in which like reference numerals designate like elements and wherein:

FIG. 1 is a partial cross-sectional view of a human heart depicting a failed mitral valve wherein the chordae tendineae have torn and the leaflet portion of the valve is disconnected from the papillary muscle and wherein a stabilizer, of the method of the present invention, has been releasably secured to the outer wall of the heart at the base of the papillary muscle;

FIG. 2 is a partial cross-sectional view of the heart of FIG. 1 showing a portion of the method and apparatus of the present invention whereby an introducer is passed through the stabilizer and heart wall and up through the papillary muscle of the failed mitral valve;

FIG. 3 is a partial cross-sectional view of the heart of FIG. 2 showing a portion of the method and apparatus of the present invention whereby a leaflet clamp has been fed through the introducer and is positioned just prior to clamping the free end of the leaflet;

FIG. 4 is a partial cross-sectional view of the heart of FIG. 3 showing a suture driver device of the method and apparatus of the present invention being coupled to the proximal end of the clamp after the flail leaflet has been clamped;

FIG. 5 is a partial cross-sectional view of the heart of FIG. 4 showing a suture of the method and apparatus of the present invention that has been passed through the free end of the leaflet, with the clamp already removed from the introducer, and whereby the ends of the suture are available through the introducer;

FIG. 6 is a partial cross-sectional view of the heart of FIG. 5 showing the mitral valve repaired using the method and apparatus of the present invention whereby the free ends of the suture have been passed through a securement ring that is positioned against the exterior side of the heart; and wherein the free ends of the suture are momentarily coupled to a strain gauge;

FIG. 6A is an enlarged isometric view of the securement ring of FIG. 6, showing internal channels with teeth for securing the free ends of the suture at a desired tension level, as well as a corresponding locking cap that fits snuggly within the securement ring;

FIG. 6B is an enlarged cross-sectional view of the securement ring and locking cap of FIG. 6A taken along line 6B-6B of FIG. 6A;

FIG. 7 is an enlarged partial cross-sectional view of the working end of the clamp of the method and apparatus of the present invention with the free end of the leaflet positioned between the clamp members;

FIG. 7A is an enlarged cross-sectional view of the working end of the clamp of the method and apparatus of the present invention showing the first member of the clamp displacing the free end of the leaflet toward the second member of the clamp;

FIG. 8 is an enlarged cross-sectional view of the working end of the clamp of the method and apparatus of the present invention showing the leaflet being clamped between the two clamp members;

FIG. 9 is an enlarged cross-sectional view of the working end of the clamp of the method and apparatus of the present invention showing the free end of the leaflet being punctured by a puncturing member;

FIG. 10 is an enlarged cross-sectional view of the working end of the clamp and whereby the suture is driven through one of the clamp members, through the hole in the free end of the leaflet and down through the other clamp member;

FIG. 11 is an enlarged cross-sectional view of the working end of the clamp and whereby the puncturing member has been withdrawn;

FIG. 12 is an enlarged cross-sectional view of the working end of the clamp and whereby the clamp members are drawn apart, thereby allowing these members to be displaced separately through the introducer without snagging the suture that has passed through the leaflet;

FIG. 13 is an enlarged cross-sectional view of the first member of the clamp and the suture passing through the leaflet after the second member of the clamp has already been withdrawn from the introducer (not shown);

FIG. 14 is a partial cross-sectional view of the overall invention depicting how the suture driver device couples to the clamp in order to drive the suture through one member of the clamp, through the aperture in the leaflet and back through the other member of the clamp; and

FIG. 14A is an enlarged partial cross-sectional view of an alternative port design of the suture driver device.

### DETAILED DESCRIPTION OF THE INVENTION

The method and apparatus of the present invention are directed to repairing a mitral valve by securing the leading edge of a flail leaflet segment with artificial chordae to the corresponding papillary muscle tip. This is accomplished without cardiopulmonary bypass and, ideally, without an incision, e.g., using thoracoscopic techniques. Thus, the present invention provides a new method for mitral valve repair for the pathology of flail mitral leaflet using proven techniques but utilizing a novel approach and new instrumentation. This allows for anatomic restoration without the need to stop the heart, use the heart-lung machine or making incisions on the heart. The method is a cardiac surgical procedure that involves transmural techniques. The term “transmural” is used in its broadest sense and includes, but is not limited to, transventricular procedures. Thus, the method of the present invention can be adapted to thoracoscopic techniques and may obviate the need for open incision.

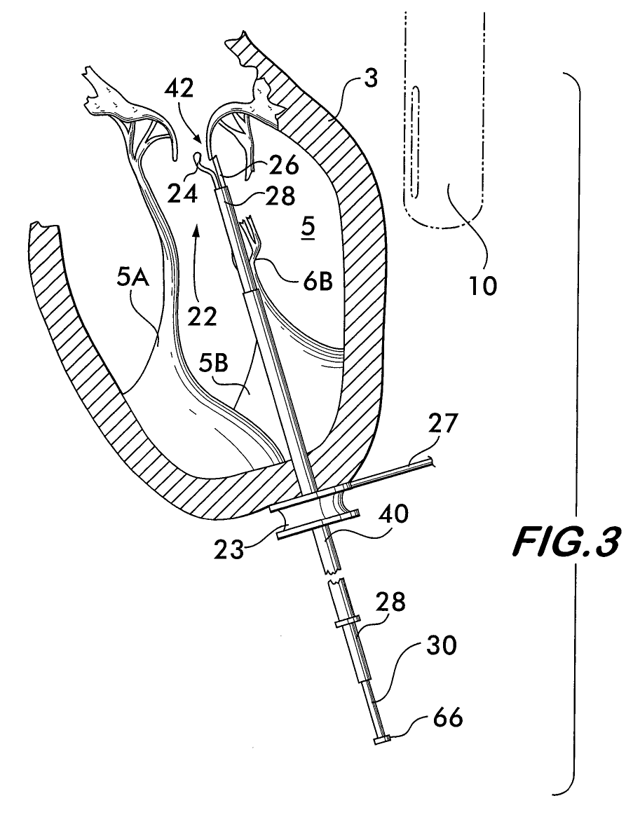

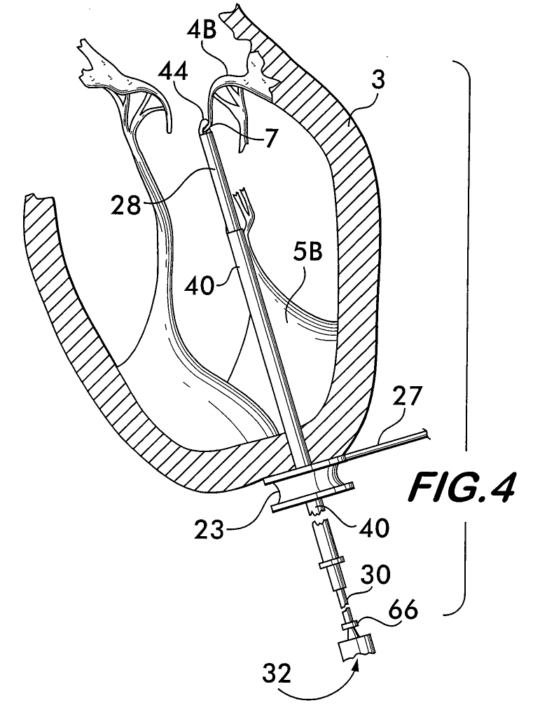

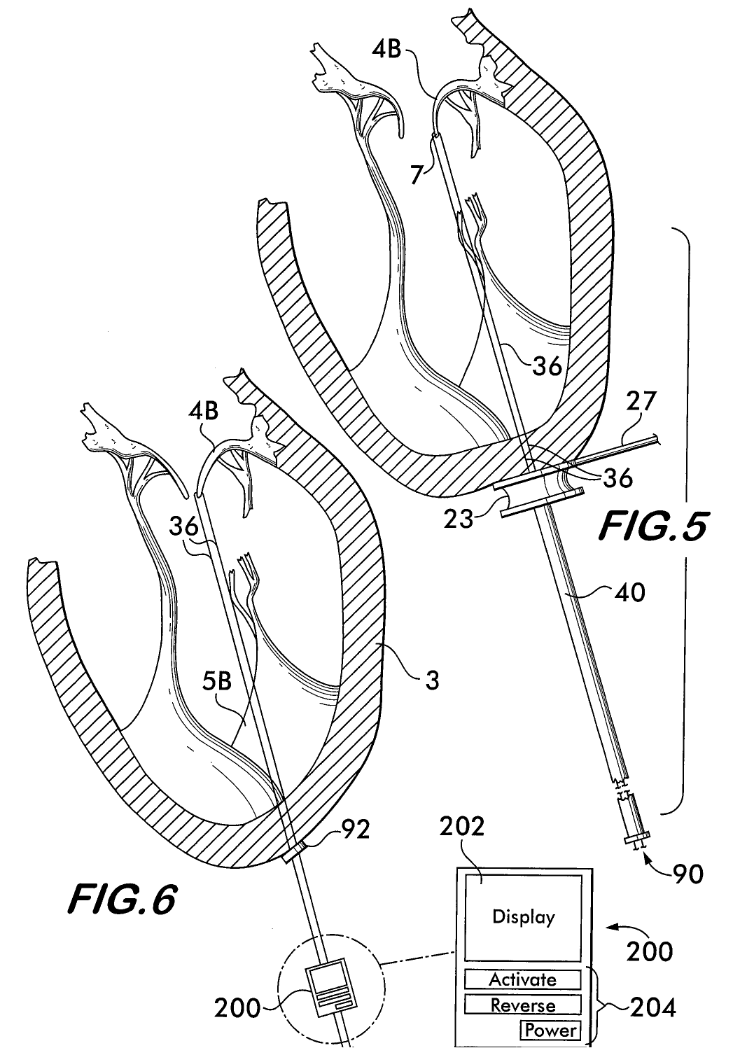

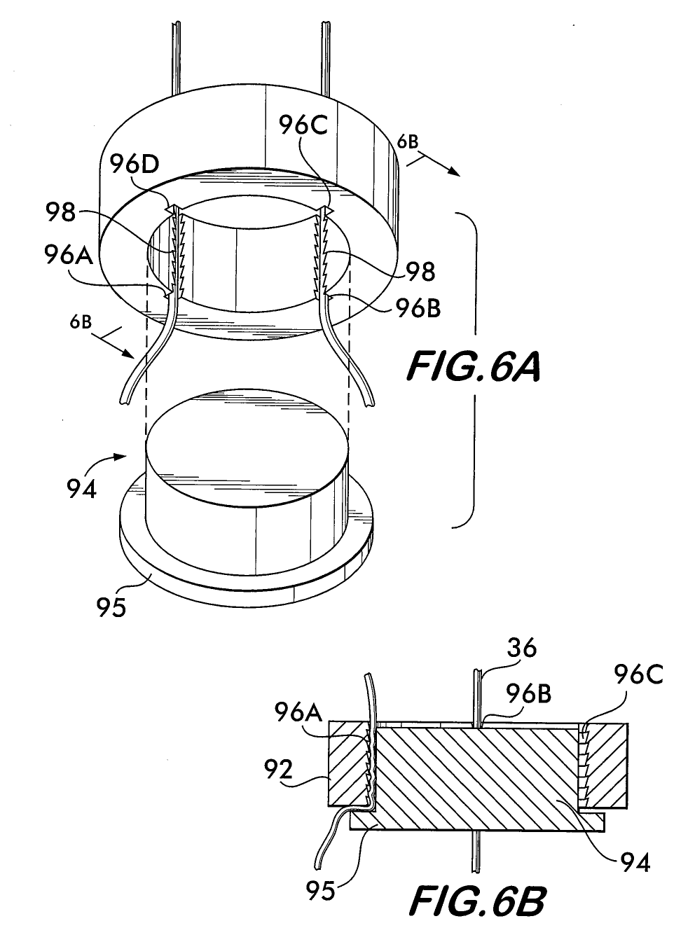

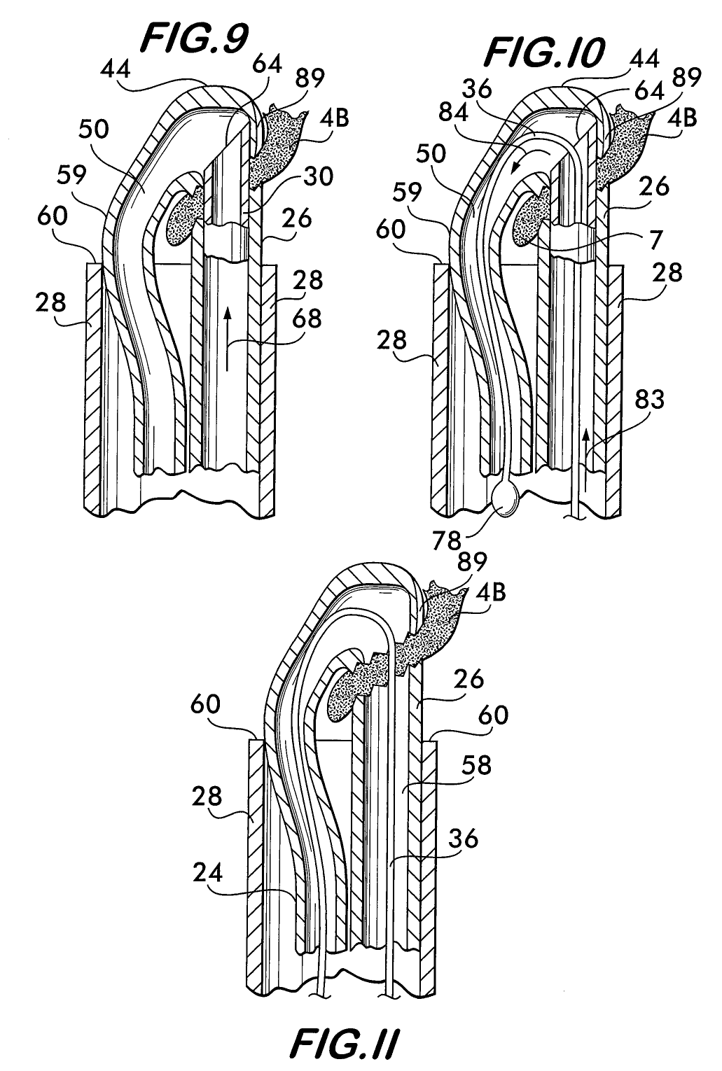

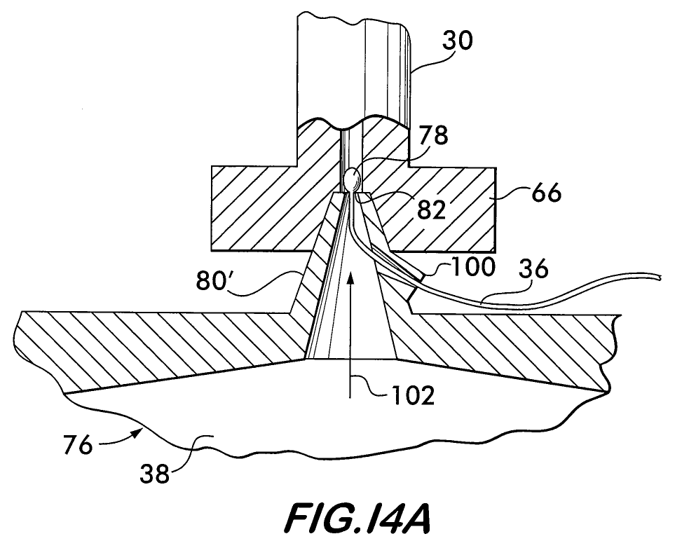

The apparatus 20 used to accomplish the method of the present invention is shown in FIG. 14. The apparatus 20 comprises a leaflet clamp 22 (comprising a first member 24 and a second member 26), a sleeve or external cylinder 28, a hollow piercing member 30 (e.g., a needle) and a suture driver device 32 (e.g., a syringe 34 comprising a suture 36 and a biocompatible fluid 38, e.g., saline solution). As will be discussed in detail later, the method of the present invention basically involves:
positioning the working end of the leaflet clamp 22, using an introducer or sheath 40, within the heart 3 through the papillary muscle from which the flail leaflet has partially detached, suffered chordal rupture or chordal defect (e.g., elongated chordal defect); see FIG. 3;
grasping the free end of the flail leaflet with the clamp 22; see FIG. 4;
piercing the clamped leaflet with the piercing member 30; see FIG. 9;
passing a suture through the clamp 22/piercing member 30 using the suture driver device 32; see FIG. 5;
removing the clamp 22, thereby leaving a suture that passes through the flail leaflet and its corresponding papillary muscle; see FIG. 6; and
securing the ends of the suture against the exterior heart wall using a securement ring/lock cap such that the leaflet is connected to its corresponding papillary muscle (see FIGS. 6-6B).

In describing the method and apparatus of the present invention, failure of the posterior leaflet is depicted by way of example only and it should be understood that other leaflet segments may be involved as well and that the method and apparatus are not limited, in any way, to the posterior leaflet. Moreover, the term “flail leaflet” is used in its broadest sense to mean any type of damage involving the leaflet, not just chordal rupture, e.g., partial chordal detachment, chordal rupture or some chordal defect (e.g., elongated chordal defect).

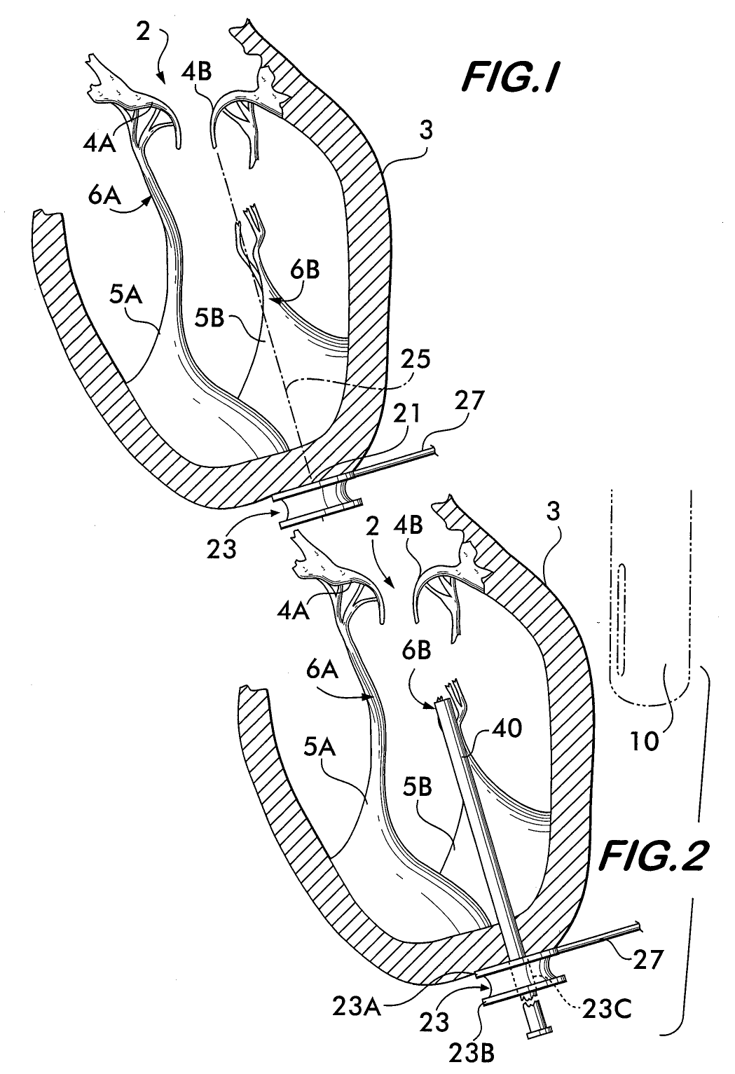

To begin the method of the present invention, the heart is exposed via stemotomy, left anterior thoracotomy or thorascopy (not shown) and the pericardium is opened. A transesophogeal ultrasound probe 10 (FIGS. 2-3) is used by the surgeon to view the interior of the heart 3, including the mitral valve 2. Next, the entry point on the heart wall corresponding to the base of the papillary muscle 5B needs to be determined, hereinafter, “the base location 21.” This is accomplished using a short focal length color Doppler epivascular ultrasound probe (not shown) which includes a needle guide channel (not shown). Once the base location 21 is determined, the surgeon then couples the epivascular ultrasound probe to a suction stabilizer 23 (FIGS. 1-5) which is then applied to the base location 21. The stabilizer 23 stabilizes the base location 21 of the heart wall for supporting the epivascular ultrasound probe and entry of the introducer 40 and clamp 22, as will be discussed later. As can be seen from FIG. 2, the stabilizer 23 comprises a housing having a first support surface 23A that contacts the heart wall, a second support surface 23B, a central passageway 23C positioned between these surfaces and an arm or extension 27 integrally formed with the first support surface 23A; the central passageway 23C may contain an access seal (not shown).

The surgeon then applies the suction stabilizer 23, along with the epivascular ultrasound probe, to the base location 21. The direction of the longitudinal axis (25, see FIG. 1) of the papillary muscle must be determined next using 2-D echo imaging with the epivascular ultrasound probe. Important epicardial, intramural and papillary blood vessels are identified with Doppler interrogation and avoided. Determination of the direction of the longitudinal axis (hereinafter “the direction 25”) of the papillary muscle 5B permits defining the passage through the papillary muscle's apex.

With the base location 21 and the direction 25 determined and with the stabilizer 23 applied to the heart wall, the stabilizer 23 suction is activated and the stabilizer arm or extension 27 is made rigid (e.g., securing or anchoring the arm/extension 27 to a fixed object), thereby fixing the heart 3 and apparatus in preparation for the Seldinger technique insertion of a finder needle and guidewire, such as described in U.S. Pat. Nos. 7,077,801 (Haverich) or 7,063,679 (Maguire, et al.), by way of example only, and both of which are incorporated by reference herein. In the Seldinger technique, a needle and subsequent guidewire (neither of which are shown) pass through the epivascular ultrasound probe, the stabilizer 23, the ventricular wall and central axis 25 of the papillary muscle 5B and emerge from the tip of the papillary muscle 5B into the ventricular chamber. The epivascular ultrasound probe and needle are removed; dilators (not shown) and the specialized introducer 40 are inserted over the guidewire. The guidewire is then removed and the introducer 40 is locked into the stabilizer 23, as shown in FIG. 2, providing a stable access platform for subsequent intracardiac instrumentation.

Under conditions of systemic heparinization and by continuously flushing the devices with heparinized saline, trans-ventricular, trans-papillary introduction of the short freestanding introducer 40 with a water-tight, as well as air-tight, access seal, affixed to the stabilizer 23, is accomplished. Similar introducers or sheaths of anti-thrombotic plastic are currently in use in the cath lab (i.e., a specialized radiologic suite where cardiac catheterization is performed) for arterial access, but this introducer 40 is considerably shorter and the seal is designed to withstand the greater pulse pressure differential and unbuffered dP/dT that exists in the left ventricle.

With the introducer 40 in place, the surgeon now inserts the clamp 22 through the introducer 40 as shown in FIG. 3 and using the transesophogeal ultrasound probe 10 positions a working end 42 of the clamp 22 so that the flail leaflet 4B is located between the first and second members 24 and 26. A discussion of the working end 42 of the clamp 22 follows.

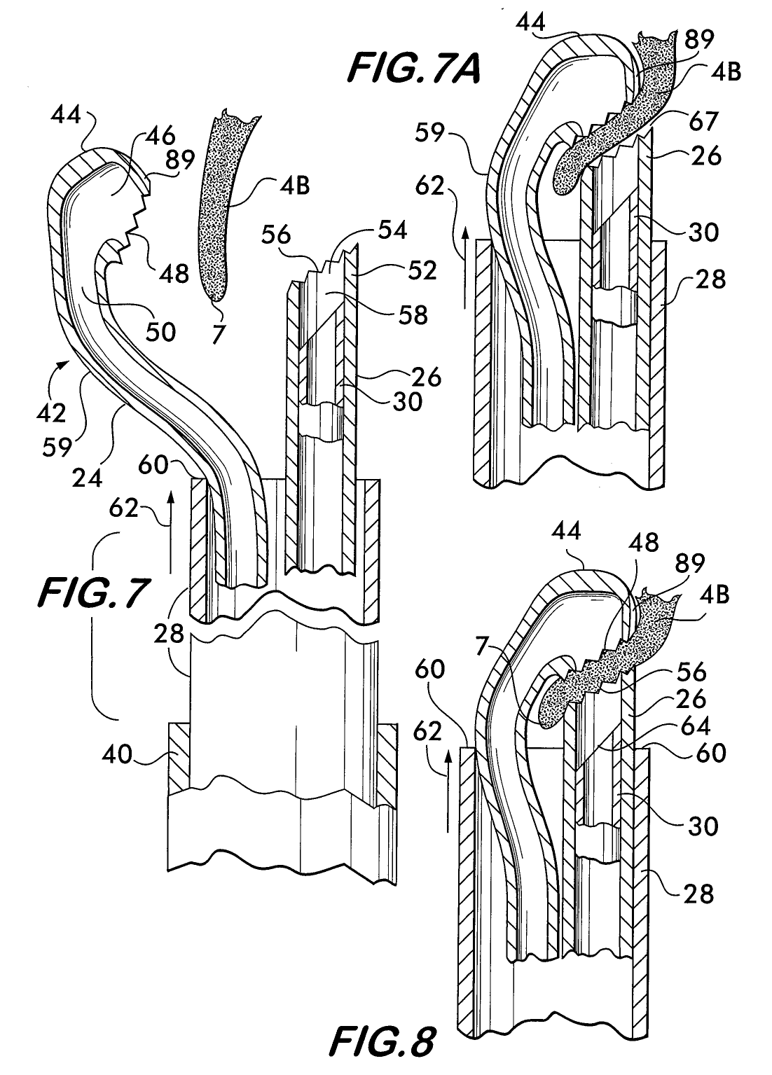

As can be seen most clearly in FIG. 7, the working end 42 of the clamp 22 comprises the first member 24 having a curved distal end 44 with an opening 46 having teeth or serrations 48 along its periphery. The first member 24 comprises a channel 50 for permitting passage of the suture 36, as will be discussed later. The working end 42 of the clamp 22 also comprises the second member 26 having a straight distal end 52 with an opening 54 also having teeth or serrations 56 along its periphery. The second member 26 (e.g., a substantially straight structure) also comprises a channel 58 for permitting passage of the hollow piercing member 30. It should be understood that the channels 50 and 58 are continuous through the members 24 and 26 and include entry or exit apertures at their respective proximal ends (not shown) thereof to allow the surgeon to introduce or remove instruments (e.g., the hollow piercing needle 30, suture 36, etc.) therefrom. The first and second members 24/26 may comprise a spring steel material; as a result, with the distal end 44 of the first member 24 having a curved configuration (including bend 59), the displacement of the external cylinder 28 in the direction 62 (FIGS. 7-7B) causes its upper end 60 to ride along the outside surface of the first member 24. Contact of the upper end 60 with the bend 59 causes the distal end 44 to contact the leaflet 4B and move it towards the second member 26, as shown in FIG. 7A; as shown by the gap 67, FIG. 7A depicts a “light control” of the leaflet 4B just prior to clamping it. Further displacement of the sleeve 28 in the direction 62 causes the respective distal ends 44 and 52 to clamp the leaflet 4B therebetween, as shown in FIG. 8. Thus, in view of the previous discussion, and following the progression of FIGS. 7-8, the surgeon initially manipulates the proximal ends (not shown) of the clamp members 24 and 26 to position the free end 7 of the flail leaflet 4B between the first and second members 24/26 as shown in FIGS. 3 and 7; the surgeon views this location using the transesophogeal ultrasound probe 10. Once the surgeon has properly positioned the free or leading edge 7 of the flail leaflet 4B between the two clamp members 24 and 26 (see FIG. 7), the surgeon gently grasps the free end 7 of the flail leaflet 4B in diastole (FIG. 7A) by partially advancing the external cylinder 28 in the direction of arrow 62. When the correct position is confirmed by echo images, clamping the leaflet free end 7 is completed by further advancement of the external cylinder 28 (in the direction of arrow 62, see FIG. 8), which also assures alignment of the respective distal ends 44 and 52 and the respective channels 50 and 58 in preparation for leaflet 4B puncturing and suture 36 advancement.

The hollow piercing member 30, if not already positioned inside the second clamp member 26, is then passed through the channel 58. The hollow piercing member 30 (e.g., a needle) comprises a sharp tapered edge 64. With the free end 7 of the flail leaflet 4B secured between the teeth/serrations 48/56, the surgeon applies pressure to the proximal end 66 (FIG. 3) of the hollow piercing member 30 in the direction of arrow 68 as shown in FIG. 9, thereby piercing the free end 7 of the flail leaflet 4B.

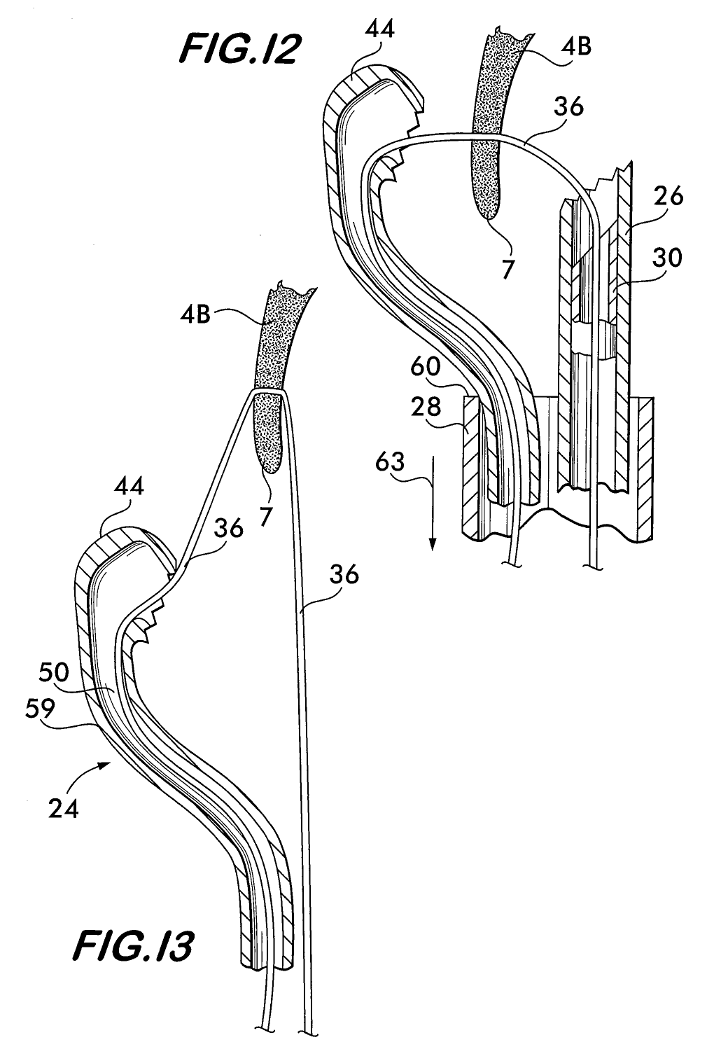

At this point, the suture driver device 32 is then coupled to proximal end 66 of the hollow piercing member 30, as shown in FIG. 4. FIG. 14 provides a more detailed view of an exemplary suture driver device 32. In particular, the device 32 comprises a syringe 34 and piston 70 and an integral stem 72. A driving side 74 of the piston 70 forms a movable wall of a chamber 76 in the syringe 34 that contains the suture 36 (e.g., 5-0 Goretex suture) and is filled with the biocompatible fluid 38. One end 78 of the suture 36 is weighted and is initially positioned at the delivery port 80 of the syringe 34; the weight acts to initially block the opening 82 in the port 80 (e.g., see FIG. 14A; alternatively, the weighted end 78 may be arranged to be internal of the port 80, in which case, the opening 82 is sized to permit passage of the weighted end 78). During insertion, the weighted end 78 and delivery port 80 are inserted into the open end 81 of the hollow piercing member 30. When the suture driver device 32 is activated by compressing the stem 72 into the syringe 34, the piston 70 compresses the fluid 38, thereby displacing the weighted end 78 by a fluid 38 stream up through the hollow piercing member 30 (see arrow 83 in FIG. 10) out of the tapered end 64 and through the channel 50 (shown by the arrow 84 in FIG. 10) in the first member 24. The fluid stream from the suture driver device 32 causes the weighted end 78 of the suture 36 to travel completely through the channel 50 so that the weighted end 78 emerges from an opening 86 (FIG. 14) in the proximal end 88 of the first member 24.

To prevent clogging the port 80 by the suture 36 as the suture driver device 32 is activated, the suture 36 is coiled (see FIG. 14) and treated with an adhesive (e.g., bonewax) when initially disposed in the chamber 76 against the piston 70. Thus, because of the applied bonewax, when fluid 38 is drawn into the chamber 76, and the coiled suture 36 is immersed in the fluid 38, the suture 36 remains coiled and only the pulling force of the weighted end 78 of the suture 36 (when the suture driver device 32 is activated) causes the coiled suture portions to separate and thereby avoid fouling or clogging the port opening 82.

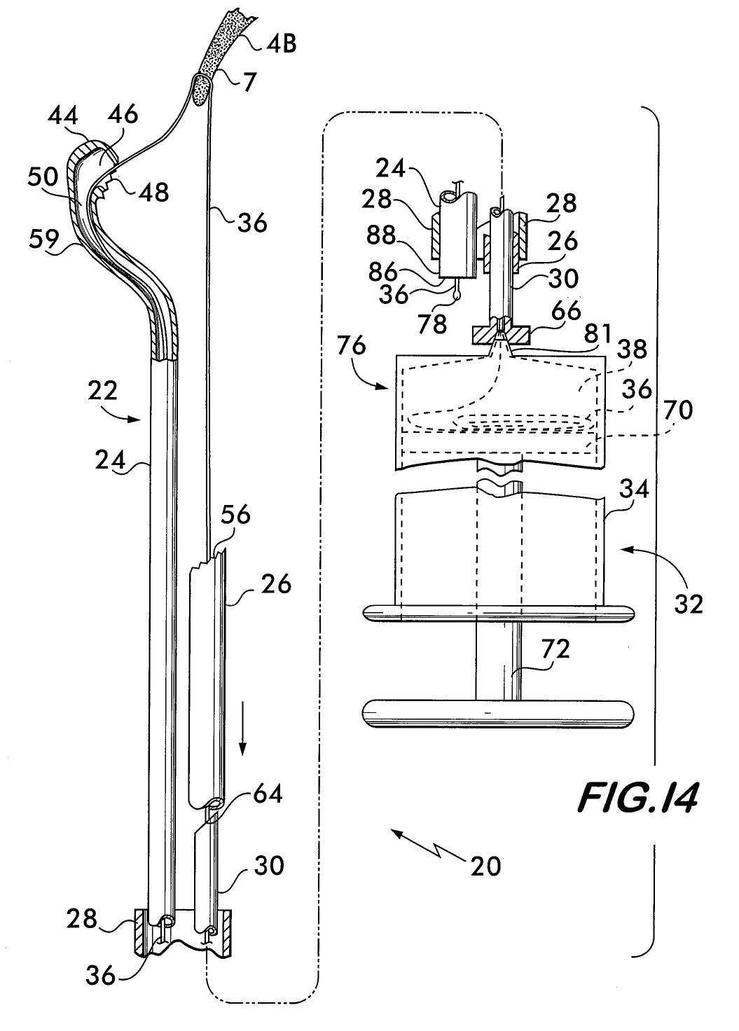

At this point of the method of the present invention, a suture 36 has been effectively passed through the free end 7 of the leaflet 4B. Before the ends of the suture 36 can be tied off, or otherwise secured, at this point, it only remains to remove the leaflet clamp 22 from the heart 3. FIGS. 11-13 show the sequence of performing this removal. In particular, the hollow piercing member 30 is removed (FIG. 11) from the second clamp member 26 by sliding it out. This can be accomplished by displacing the suture driver device 32 away from the second member 26; alternatively, the suture driver device 32 can first be disengaged from the open end 81 of the hollow piercing member 30 and then the hollow piercing member 30 removed from the second member 26. In either case, removal of these items from the second member 26, is accomplished while leaving the suture 36 in place by not snagging it within the clamp 22 components during their removal. As shown in FIG. 12, with the hollow piercing member 30 removed, the clamp 22 is opened by displacing the sleeve 28 in the direction of arrow 63, which permits the first member 24 to swing away from the second member 26, thereby releasing the free end 7 of the leaflet 4B. The second clamp member 26 is then displaced in the direction of arrow 63, thereby being retracted within the external cylinder 28 and removed therefrom. This leaves the first clamp member 24 and the suture 36 protruding out of the sleeve 28 (FIG. 12, the external cylinder 28 has been slid in the direction of the arrow 63 and is therefore no longer visible in FIG. 13) and the surgeon now needs to retract the first member 24 therein.

As can be seen most clearly in FIG. 13, the suture 36 rides along the tip of the distal end 44. An indentation or groove 89 (FIGS. 7-11 and 13) is provided in the tip of the distal end 44 to maintain the suture 36 at the tip of the distal end 44 during removal of the first clamp member 24. This prevents the suture 36 from becoming snagged or caught in the teeth/serrations 48, especially when the first clamp member 24 is being retracted within the sleeve 28, as discussed next.

The first clamp member 24 is retracted within the external cylinder 28 (as mentioned earlier, the contact of the protuberance 59 with the top edge (not shown) of the external cylinder 28 causes the first member 24 to displace to the right, with reference to FIG. 13). Once retracted within the external cylinder 28, the cylinder 28 is removed from the introducer 40. Thus, with the clamp 22 withdrawn, the result is a single suture 36 now is looped through the free end 7 of the leaflet and the ends 90 (FIG. 5) of the suture 36 protrude out of the proximal end of the introducer 40. More particularly, with the length of the suture 36 passing through the introducer/sheath 40, the ends 90 of the suture 36 are brought to the exterior of the heart 3 (and the patient) where the ends 90 are temporarily secured to the surgical drapes (not shown). Additional sutures can be established by re-introduction of the clamp 22 into the introducer/sheath 40 beside the previous sutures, and the above-described method is repeated with the clamp 22 and suture driver device 32. Typically, three to eight sutures might be required to completely support the flail leaflet 4B, depending on the extent of pathology. After implantation of all of the sutures 36, with the pairs of ends all exterior to the heart 3, the sheath 40 is removed (FIG. 5), and the sutures 36 are individually adjusted to the appropriate length with real time echocardiographic guidance for optimal line of leaflet co-aptation. Furthermore, if no more sutures are to be passed through the leaflet 4B, the stabilizer 23 is also removed.

To complete the mitral valve repair, as shown in FIG. 6, a securement ring 92 and corresponding locking cap 94 (FIGS. 6A-6B) are provided. In particular, the securement ring 92 comprises a plurality of channels having locking teeth therein. By way of example only, four such channels 96A-96D are shown in FIG. 6A, located 90 degrees from each other, and two of which, channels 96A and 96B, are depicted with the suture 36 disposed therein. The locking teeth 98 in each channel prevent the suture 36 portions from pulling out once they are positioned in these channels. Once each suture 36 portion is positioned within a respective channel, the locking cap 94 is secured inside the securement ring 92 as shown in FIGS. 6A-6B, thereby locking the suture portions against the exterior heart wall. Besides securing the free ends 90 of the suture 36 against the ring 92, an integral rim 95 of the locking cap 94 also provides a surface for grasping the cap 94 should it ever be necessary to obtain access to the sutures 36 in the future during surgery. The ring 92/cap 94 assembly is designed to both complete hemostasis and distribute the tension on the new suture chordae in systole; both the ring 92 and cap 94 comprise biocompatible material. The end result is that the leaflet 4B is now coupled to its corresponding papillary muscle 5B.

It should be understood that prior to inserting the suture portions into the securement ring 92 channels, as shown in FIG. 6, the suture portions are first coupled to a strain gauge device 200. The strain gauge device 200 permits the surgeon to measure the tension applied to the suture 36, and adjust it accordingly, before locking the suture portions into the securement ring 92 channels. In particular, the free ends 90 of the suture 36 are passed through the securement ring 92. Next, the free ends 90 are coupled to the strain gauge device 200. The surgeon measures the tension being applied to the free ends 90 and can adjust that tension accordingly. Once the surgeon is satisfied with the tension on the suture portions 36, the surgeon positions the suture portions in respective securement ring channels (e.g., 96A-96D), thereby locking the suture within the securement ring 92 at the desired tension level. These tension measurements are important because it may not be ideal to apply the same tension to the repaired leaflet chordae as are applied to the undamaged chordae of the other leaflet. In fact, such higher “tension” may have led to the flail leaflet in the first place. Thus, by using strain gauges/load cells as part of the method of the present invention, the surgeon can further assess repair physiology and thereby provide the most effective repair.

By way of example only, the strain gauge device 200 may comprise a strain gauge or load cell, such as the S251 miniature platform load cell by Strain Measurement Devices of Meriden, Conn. The strain gauge device 200 may also comprise a display 202 for displaying the tension values. The device 200 may also comprise a stepper motor for applying incremental, increasing or decreasing, steps of tension for more precise control of the tension. Corresponding keys 204 provide such control to the surgeon.

It should be appreciated that by using the structure of the apparatus 20, there are no small components (e.g., known clamps and/or cutters that have articulating, hinged, journaled, etc,. components that utilize screws or other fasteners that can also dislodge) which enter the heart that can dislodge and form an embolism; rather, the components of the apparatus 20 form continuous members with no hinged or articulating parts that could break off.

The overall diameter of the clamp 22 must allow easy passage through the introducer 40 (e.g., an 8 to 10 french sheath or approximately 2.7 mm). This allows multiple subsequent passes of the external cylinder 28 within the sheath 40 along-side previously placed sutures 36. As mentioned earlier, the clamp members 24 and 26 may comprise spring steel such that they open when the clamp 22 is withdrawn (e.g., sliding a control ring (not shown) on the body of the device back). The respective distal ends 44 and 52 of the clamp members 24/26 close gently with partial advancement of the external cylinder 28, and firmly with complete advancement, which also aligns the respective channels 50 and 58. The channels 50 and 58 may contain heparinized saline flush and are capped (not shown) at their proximal ends until the leaflet 4B is grasped. The cap is removed for hollow piercing member 30/suture 36 passage, and can be replaced after completed suture placement, clamp 22 withdrawal and channel flushing for subsequent passes.

It should be noted that, although not shown, the preferred inner surface of the external cylinder 28 is oval or elliptical. This preferred shape prevents the first and second members 24 and 26 from passing each other during displacement of the external cylinder 28 (in the direction 62) which could cause misalignment of the distal ends 44 and 52 which could tear the leaflet 4B during clamping and could also cause misalignment of the respective channels 50 and 58.

An alternative port 80′ design is shown in FIG. 14B. In this port 80′, a suture port 100 is provided to permit passage of the suture 36 externally of the suture driver device 32. This eliminates the need to stow the suture 36 within the suture driver device 32, as well as treat the suture with an adhesive, prior to use. In this embodiment, the free end (not shown) of the suture 36 is first passed through the port opening 82 and then through the suture port 100 until the weighted end 78 of the suture 36 comes to rest against the port opening 82, as shown in FIG. 14A. Because of the angled design of the suture port 100, when the suture driver device 32 is activated (as discussed with regard to FIG. 14), the fluid 38 drives the weighted end 78 up the hollow piercing member 30 (in the direction of the arrow 102), with minimal loss of fluid 38 through the suture port 100.

Another alternative embodiment (not shown) also permits passage of the suture externally of the suture driver device 32 but without the need for a suture port. In this alternative embodiment, a channel or groove on the outside surface of the port 80 is provided. With the weighted end 78 of the suture 36 disposed on the port opening 82 (as shown in FIG. 14A), once the suture driver device 32 is activated, the suture 36 is also drawn upward into the hollow piercing member 30 by the fluid stream and the suture 36 feeds upward through the channel or groove. Using this embodiment, it also eliminates the need to stow the suture 36 within the suture driver device 32, as well as treat the suture with an adhesive, prior to use.

As mentioned earlier, three to eight sutures may be required to completely support the flail leaflet 4B. Thus, as a result, the securement ring 92 may include sixteen locking channels since each suture looped through the leaflet 4B has two portions. Again, the number of locking channels is by way of example only and is not limited to those shown or discussed.

Although less preferred, an alternative to the suture driver device 32 is to have the suture 36 comprise a sharp-tipped wire (not shown) swedged to one end of the suture 36. “Swedging” is the same technology that attaches sutures to needles in open surgery. Thus, the sharp-tipped wire is manually displaced through the leaflet clamp 22, while towing the suture 36. Once the wire portion emerges from the proximal end 88 of the first member 24, the wire portion can be severed from the suture 36. A funnel enlargement of the tip opening 46 of the channel 50 directs transfer of the point of the wire into the first member channel 50 after it pierces the leaflet 4B.

Systemic heparin is reversed. The transpapillary muscle tract of the apparatus 20 and subsequent sutures are extrinsically compressed by ventricular pressure in systole. This compression and the tract length should minimize bleeding. Systemic beta blockade and avoiding early post-op hypertension are sensible precautions.

Performing the maneuvers of this method should be well tolerated by the beating heart because they do not worsen, but rather progressively improve the severe valvular regurgitation as the operation progresses before ultimately eliminating the leak at completion. Planned evolution of this operation after initial development and testing is via a standard median sternotomy to allow conversion to standard operation, then as experience is gained and with minimally modified instrumentation, through a small anterior left thoracotomy incision to gain access to the off-apex base of the papillary muscle. Ultimately, it is desirable to develop the instruments and techniques to refine this repair into a minimally invasive left-sided thoracoscopic procedure without surgical incision.

Successful application of this technique would result in a natural appearing mitral valve resulting from restoration of normal anatomy. This is in contrast to other proposed off pump techniques derived from the Alfieri stitch. Failure of this technique should not result in embolization, worsening of baseline pathology or preclude subsequent cardiac surgeries including subsequent mitral valve operations. This technique might also be of value in other situations such as repair of leaking prosthetic valves or patches, tacking down of mobile intra-luminal or intra-chamber flaps or implants or leads, or whenever external suture support of other intra-cardiac structures or prostheses is required.

While the invention has been described in detail and with reference to specific examples thereof, it will be apparent to one skilled in the art that various changes and modifications can be made therein without departing from the spirit and scope thereof.

What is claimed is:

1. A method for repairing the mitral valve of a heart wherein at least one leaflet has suffered a defect with respect to its papillary muscle, said method comprising:
introducing a clamp transmurally into the beating heart and through the papillary muscle;
grasping a portion of the leaflet with said clamp;
piercing a hole in the leaflet;
inserting a suture, having a first end, through said clamp and through said hole, said first end being displaced through said clamp instrument to emerge from a proximal end of said clamp;
removing said clamp from the beating heart; and
securing said first end, and a second end, of said suture against an exterior wall of the beating heart.

2. The method of claim 1 wherein said step of introducing a clamp transmurally comprises:
using a probe to select an entry spot on a heart wall corresponding to the base of the papillary muscle; and
using said probe to select a direction for inserting said introducer along a center or longitudinal axis of the papillary muscle.

3. The method of claim 2 wherein said step of using said probe to select a direction comprises:
coupling said probe to a stabilizer;
applying said stabilizer to the heart wall at said selected entry spot; and
selecting said direction using said probe.

4. The method of claim 3 further comprising:
activating said stabilizer to stabilize a vicinity around said entry spot;
using the Seldinger technique to pass a needle and guidewire through said stabilizer and probe at said selected entry spot along said selected direction to emerge from a tip of the papillary muscle into the heart's ventricular chamber;
removing said needle and probe;
inserting said introducer over said guidewire;
removing said guidewire; and
locking said introducer to said stabilizer.

5. The method of claim 1 wherein said probe comprises an epivascular ultrasound probe.

6. The method of claim 1 wherein said step of piercing a hole in the leaflet comprises inserting a hollow piercing member through said clamp, said hollow piercing member having a leading edge that punctures the leaflet.

7. The method of claim 6 wherein said step of inserting a suture through said clamp comprises using a fluid stream to drive a suture having a weight at its first end through said hollow piercing member while maintaining said second end of said suture at said proximal end of said clamp.

8. The method of claim 7 wherein said step of using a fluid stream to drive a suture comprises disposing said suture in a syringe having a port that can couple to a proximal end of said hollow piercing member, said syringe comprising a chamber filled with a biocompatible fluid and with said suture, said first end being initially disposed at said port such that when said syringe is activated, said weight is driven through said clamp.

9. The method of claim 7 wherein said step of using a fluid stream to drive a suture comprises:
providing a syringe having a first port that can couple to a proximal end of said hollow piercing member and having a second port, in fluid communication with said first port, said syringe comprising a chamber filled with a biocompatible fluid; and
passing said second end through said first port and through said second port until said first end is disposed at said first port such that when said syringe is activated, said weight is driven away from said first port while pulling said suture through said second port and through said first port and through said clamp.

10. The method of claim 1 wherein said step of introducing a clamp comprises introducing first and second members that cooperate to form a clamp, said first member comprising a first opening at a first distal end that is communication with a first channel that runs the length of said first member, and wherein said second member comprises a second opening at a second distal end that is communication with a second channel that runs the length of said second member, said first and second openings being aligned when said first and second distal ends grasp the leaflet.

11. The method of claim 10 wherein said step of piercing a hole in the leaflet comprises inserting a hollow piercing member through said second channel, said hollow piercing member having a leading edge that punctures the leaflet and enters said first opening.

12. The method of claim 11 wherein said step of inserting a suture through said clamp comprises using a fluid stream to drive a suture having a weight at its first end through said hollow piercing member and through said first channel while maintaining said second end of said suture at said proximal end of said clamp.

13. The method of claim 12 wherein said step of using a fluid stream to drive a suture comprises disposing said suture in a syringe having a port that can couple to a proximal end of said hollow piercing member, said syringe comprising a chamber filled with a biocompatible fluid and with said suture, said first end being initially disposed at said port such that when said syringe is activated, said weight is driven through said clamp.

14. The method of claim 12 wherein said step of using a fluid stream to drive a suture comprises:
providing a syringe having a first port that can couple to a proximal end of said hollow piercing member and having a second port, in fluid communication with said first port, said syringe comprising a chamber filled with a biocompatible fluid; and
passing said second end through said first port and through said second port until said first end is disposed at said first port such that when said syringe is activated, said weight is driven away from said first port while pulling said suture through said second port and through said first port and through said clamp.

15. The method of claim 1 wherein said step of securing said first end and a second end of said suture against an exterior wall of the beating heart comprises:
passing said first and second ends through a biocompatible ring having a plurality of channels within an inner surface thereof;
positioning said ring against the exterior wall of the beating heart;
inserting first and second portions of said suture in respective ones of said plurality of channels; and
inserting a cap within said ring to lock said first and second ends between said inner surface and said cap.

16. The method of claim 15 wherein said step of passing said first and second ends through a biocompatible ring further comprises:
coupling said first and second ends to a strain gauge to determine any tension applied through said suture;
adjusting the tension of said suture until a desired tension is achieved; and
de-coupling said first and second ends from said strain gauge.

17. A method for repairing the mitral valve of a heart wherein at least one leaflet has suffered a defect with respect to its papillary muscle, said method comprising:
(a) introducing a clamp transmurally into the beating heart and through the papillary muscle;
(b) grasping a portion of the leaflet with said clamp;
(c) piercing a hole in the leaflet;
(d) inserting a suture, having a first end, through said clamp and through said hole, said first end being displaced through said clamp to emerge from a proximal end of said clamp;
(e) maintaining a second end of said suture external to the beating heart;
(f) removing said clamp from the beating heart;
(g) repeating steps (a)-(f) to establish a plurality of first ends that emerge from a proximal end of said clamp and a plurality of second ends that are maintained external to beating heart; and
(h) securing said plurality of first ends and said plurality of second ends against an exterior wall of the beating heart.

18. An apparatus for repairing the mitral valve of a heart wherein at least one leaflet has suffered a defect with respect to its papillary muscle, said apparatus comprising:
a clamp comprising first and second elongated members having respective first and second distal ends for clamping the leaflet;
an external cylinder in which said clamp is slidable;
a hollow piercing member, having a leading edge that can pierce tissue, that slides within said clamp;
a suture driver device that couples to one end of said hollow piercing member, and
wherein movement of said external cylinder acts on said first and second members to open or close said clamp to grasp or release the leaflet, wherein said displacement of said hollow piercing member punctures said leaflet to form a hole therein and wherein said suture driver device drives a suture through said hollow piercing member for permitting said suture to pass through the leaflet and through said clamp for supporting mitral valve repair by connecting the leaflet to the papillary muscle.

19. The apparatus of claim 18 wherein said first member comprising a first opening at said first distal end that is communication with said first channel that runs the length of said first member, and wherein said second member comprises a second opening at said second distal end that is communication with a second channel that runs the length of said second member, said first and second openings being aligned when said first and second distal ends grasp the leaflet.

20. The apparatus of claim 19 wherein said first distal end is curved and wherein said first opening is located at a first tip of said first distal end.

21. The apparatus of claim 20 wherein said second member comprises a substantially straight structure and wherein said second opening is located at a second tip of said second distal end.

22. The apparatus of claim 18 wherein said suture driver device comprises a syringe having a port that can couple to a proximal end of said hollow piercing member, said syringe comprising a chamber filled with a biocompatible fluid and with said suture, said suture comprising a weighted end that is initially disposed at said port such that when said syringe is activated, said weighted end is driven through said clamp.

23. The apparatus of claim 22 wherein said suture is coiled and an adhesive applied thereto for maintaining said suture in a coiled state even when said coiled suture is immersed in said biocompatible fluid.

24. The apparatus of claim 23 wherein said adhesive comprises bonewax.

25. The apparatus of claim 18 wherein said suture driver device comprises a syringe having:
a first port that can couple to a proximal end of said hollow piercing member;
a second port, in fluid communication with said first port;
a chamber in fluid communication with said first and second ports and filled with a biocompatible fluid; and
wherein, before said suture driver device is activated, said suture is passed through said first port and through said second port so that a second end of said suture is located externally of said suture driver device and said weighted end of said suture is positioned at said first port.

26. The apparatus of claim 25 wherein said second port is angled with respect to said first port such that said passage of said biocompatible fluid through said second port is minimized when said suture driver device is activated.

27. The apparatus of claim 23 wherein said hollow piercing member is positioned within said second member.

28. The apparatus of claim 21 wherein said hollow piercing member is a needle.

29. The apparatus of claim 20 wherein said first member comprises spring steel.

30. The apparatus of claim 21 wherein said second member comprises spring steel.

31. The apparatus of claim 18 further comprising a securement ring, said securement ring being positioned against an exterior wall of said heart and comprising a plurality of channels within an inner surface of said ring for receiving respective portions of sutures that have passed through the leaflet, each of one of said plurality of channels comprising a plurality of teeth for capturing a respective portion of said suture.

32. The apparatus of claim 31 further comprising a locking cap, said locking cap cooperating with said securement ring for locking said portions of said suture within said plurality of channels.

33. An apparatus for stabilizing a portion of the heart wall of a beating heart to permit the transmural introduction of surgical instruments through the heart, said apparatus comprises a housing having:
a first support surface that contacts the heart wall of the beating heart and provides a stable target for transmural penetration;
a central passageway for permitting coupling of an epivascular ultrasound probe, for the passage of instruments used for the Seldinger technique, and for the passage of an introducer therethrough; and
an extension formed with said first support surface for coupling to an externally fixed object.

34. A suture driver device for driving a suture through a surgical device that has penetrated some portion of a living being and wherein the surgical device provides a path for delivery of the suture, said suture driver device comprising a syringe having a port that can couple to the surgical device, said syringe comprising a chamber filled with a biocompatible fluid and with said suture, said suture comprising a weighted end that is initially disposed at said port such that when said syringe is activated, said weighted end is driven through said surgical device.

35. The apparatus of claim 34 wherein said suture is coiled and an adhesive applied thereto for maintaining said suture in a coiled state even when said coiled suture is immersed in said biocompatible fluid.

36. The apparatus of claim 35 wherein said adhesive comprises bonewax.

37. A suture driver device for driving a suture through a surgical device that has penetrated some portion of a living being and wherein the surgical device provides a path for delivery of the suture, said suture driver device comprising a syringe having:
a first port that can couple to a proximal end of the surgical device;
a second port, in fluid communication with said first port;
a chamber in fluid communication with said first and second ports and filled with a biocompatible fluid; and
wherein, before said suture driver device is activated, said suture is passed through said first port and through said second port so that a first end of said suture is located externally of said suture driver device and a second weighted end of said suture is positioned at said first port.

38. The apparatus of claim 37 wherein said second port is angled with respect to said first port such that said passage of said biocompatible fluid through said second port is minimized when said suture driver device is activated.

39. An apparatus for securing the free ends of a suture that have passed through an internal body part of a living being, said apparatus comprising:
a ring having an inner surface with a plurality of channels, each of said channels comprising teeth;
a corresponding plug that fits snuggly within an opening of said ring;
wherein the free ends of the suture are passed through said opening in said ring and each one of said free ends are positioned in respective ones of said plurality of channels and wherein said plug is then positioned snuggly within said opening and wherein said ring and plug are positioned against said internal body part.

40. A strain gauge device for detecting the tension applied to the free ends of a suture that has passed through the body part of a living being, said strain gauge device comprising a housing that can be coupled to the free ends of the suture, said housing comprising:
a strain gauge or load cell for detecting the strain or load applied to the suture;
a display, coupled to said strain gauge or load cell, for displaying tension values;
a stepper motor, coupled to said display, for increasing or decreasing applied tension to the suture; and
control keys coupled to said display and to said stepper motor for permitting a user to control the tension applied to the suture.
